(19) United States
(12) Patent Application Publication
(10) Pub. No.: US 2011/0130804 A1
(43) Pub. Date: Jun. 2, 2011

(54) IMPLANTABLE PULSED-RADIOFREQUENCY MICRO-STIMULATION SYSTEM

(75) Inventors: Chii-Wann Lin, Taipei (TW); Mu-Lien Lin, Taipei (TW); Shey-Shi Lu, Taipei (TW); Win-Pin Shih, Taipei (TW); Yeong-Ray Wen, Taipei (TW); Hung-Wei Chiu, Taipei (TW)

(73) Assignee: UniMed Investment Inc., Taipei (TW)

(21) Appl. No.: 12/952,673

(22) Filed: Nov. 23, 2010

Related U.S. Application Data
(60) Provisional application No. 61/265,128, filed on Nov. 30, 2009.

(51) Int. Cl. A61N 1/36

(52) U.S. Cl. 607/45; 607/46; 607/2

(57) ABSTRACT

The present invention relates to a method for treating a nervous symptom or condition in a subject with a pulsed-radiofrequency stimulation system with a low voltage to overcome the disadvantages of the known related stimulation systems.

### CROSS-REFERENCE TO RELATED APPLICATION PARAGRAPH

This application claims the benefit of U.S. Provisional Application No. 61/265,128 filed on Nov. 30, 2009, the content of which is hereby incorporated by reference in its entirety.

### FIELD OF THE INVENTION

The preset invention relates to a medical device that enables a low power consumption pulsed-radiofrequency stimulation of the nervous system or tissue.

### BACKGROUND OF THE INVENTION

Nerve cells consist of an axon for transmitting action potentials or neural impulses, and dendrites for receiving such impulses. Normally, nerves transmit action potentials from the impulse-sending axon of one nerve cell to the impulse-receiving dendrites of an adjacent nerve cell. At synapses, the axon secretes neurotransmitters to trigger the receptors on the next nerve cell's dendrites to initiate a new electrical current. In one hand, transmission of action potentials is impaired so that activation of neural impulses is required to restore normal functioning. On the other hand, action potentials are transmitted which do not serve a useful purpose; hence, blocking of unnecessary or excessive neural impulses is required to restore normal functioning.

Electrical energy to the spinal cord has been applied for the purpose of managing pain since 1960s. It is known that application of electrical field to spinal nervous tissue can effectively mask certain types of pain transmitted from regions of the body associated with the stimulated nervous tissue. Electrical energy was also used to manage the symptoms of various motor disorders, for example, tremor, dystonia, spasticity, and the like. Accordingly, electrical stimulators were developed for delivering electrical stimulation therapy in order to treat a variety of nervous symptoms or conditions, such as chronic pain (e.g. back pain), tremor, depression, Parkinson's disease, epilepsy, urinary of fecal incontinence, sexual dysfunction, or obesity.

However, conventional, non-specific stimulators may apply stimulation energy to the targeted tissue and to other non-targeted tissues beyond the intended stimulation targets. Another problem in conventional stimulators is that the amount simulation energy needed to provide the desired amount of neuro-stimulation is difficult to precisely control.

One of the electrical stimulation is continuous radiofrequency (CRF), which is a development of radiofrequency denervation based on thermo-coagulation. Recently, pulsed-radiofrequency (PRF) is used in pain management to treat especially chronic pain without thermal damages to the targeted and surrounding tissues, possibly due to the lower ohmic resistance and dissipated energy. There is a known approach in the state of the art to use an external stimulator unit with up to 40-70V pulse amplitude to ensure the effectiveness. However, such a high pulse amplitude as 40-70V is undesired because a large battery space to deliver the stimulation pulses for a long-term operation is required, and the possible re-nervation or sprouting of the nerve connections causes hyper-sensitivity to pain after nerves regeneration. Thus, repeated surgery is needed.

Accordingly, a non-destructive and implantable pulsed-radiofrequency stimulator of a small size and with a high safety is desired.

### SUMMARY OF THE INVENTION

The present invention features by a method for treating a nervous symptom or condition in a subject with a pulsed-radiofrequency stimulation system with a low voltage to overcome the disadvantages of the known related stimulation systems.

In one aspect, the present invention provides an implantable pulsed-radiofrequency stimulator for treating a nervous symptom or condition, which comprises:

a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the micro-controller via a wire for delivering the electrical stimulation pattern.

In a further aspect, the invention provides a stimulation system for treating a nervous symptom or condition, which comprises:

a remote charger for power supply; and
an implantable pulsed-radiofrequency stimulator, comprising:
a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the micro-controller via a wire for delivering the electrical stimulation pattern.

In the other aspect, the invention provides a method for treating a nervous symptom or condition in a subject comprising:

placing at least one electrode at an appropriate location on or around the nervous ganglion or the surrounding tissue of the subject as desired; and
activating the electrode(s) to general a pulsed-radiofrequency stimulation by a remove charger for power supply. In one example of the invention, the pulsed-radiofrequency stimulation at a low amplitude is generated.

In particular, the invention provides a method for pain therapy in a subject comprising:

placing at least one electrode at an appropriate location on or around the dorsal root ganglion of the subject as desired; and
activating the electrode(s) to generate a pulsed-radiofrequency stimulation by a remote charger for power supply. In one example of the invention, the pulsed-radiofrequency stimulation at a low amplitude is generated.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown. In the drawings:

FIG. 1 is a pictorial drawing of an embodiment of the electrode according to the invention referring to a stick with multiple electrodes containing one polar electrode on the top of the stick and eight contact electrodes positioned on the body of the stick, and is fixed to the nerve or tissue with four anchors.

FIG. 2 is a diagram showing the results of the von Frey behavior experiment on the treatment with or without the stimulation system according to the invention; wherein an improvement in the experiment group was found as compared with the control group (** means p<0.001, by t-test statistics).

### DETAILED DESCRIPTION OF THE INVENTION

The present invention is related to a method for treating a nervous symptom or condition with a pulsed-radiofrequency stimulation system at a low amplitude, which is of a smaller size and much safer than conventional stimulation systems. According to the invention, it is unexpectedly found that one or more electrodes exposed at an appropriate location of the nerve or tissue such as dorsal root ganglion, to generate a pulsed-radiofrequency stimulation at a low amplitude is effective in the treatment of a nervous symptom or condition, and thus it makes possible to develop an implantable small-sized stimulator without battery and with high safety. Without battery in the stimulator imposes no revisit for surgery to replace exhausted battery needed in the conventional implant system and thus causes significant decrease or elimination in pain and associated costs for patients, including economical and psychological impacts.

The present invention provides an implantable pulsed-radiofrequency stimulator for treating a nervous symptom or condition, which comprises:

a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the low power consumption controller via a wire for delivering the electrical stimulation pattern to the electrode(s).

Furthermore, the invention provides a stimulation system for treating a nervous symptom or condition, which comprises:

a remote charger for power supply; and
an implantable pulsed-radiofrequency micro-stimulator, comprising:
a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the controller via a wire for delivering the electrical stimulation pattern to the electrode(s).

Accordingly, the invention also provides a method for treating a nervous symptom or condition in a subject comprising:

placing at least one electrode at an appropriate location on or around the nervous ganglion or the surrounding tissue of the subject as desired;
activating the electrode(s) to generate a pulsed-radiofrequency stimulation by a remote charger for power supply.

In particular, the invention provides a method for pain therapy in a subject comprising:

placing at least one electrode at appropriate locations, such as, on or around the dorsal root ganglion of the subject as desired;
activating the electrode(s) to generate a pulsed-radiofrequency stimulation by a remote charger for power supply.

In one example of the invention, the electrode generates a pulsed-radiofrequency stimulation at a low amplitude, which is safer for humans or animal bodies.

The term “a nervous symptom or condition” as used herein refers to a disorder or condition in association with nervous system, including but not limited to chronic pain such as back pain, a motor disorder such as tremor, dystonia, or spasticity, cognitive disorders such as Parkinson's disease, and any other disorder such as obesity, epilepsy, depression, incontinence such as urinary or fecal incontinence, or sexual dysfunction.

According to the invention, the electrode(s) is(are) placed at an appropriate location, including a dorsal root ganglion (DRG) or a spinal ganglion (SG) of spine or a trigeminal ganglion (TG) of the 5^{th} cranial nerves, or basal ganglia (BG), hippocampus of brain, cerebellum, or autonomic nerve, or peripheral nerves. In particular, the electrode(s) is(are) exposed on or around a dorsal root ganglion.

According to the invention, the term “working parameters of radiofrequency stimulation pulses” as used herein refers to any parameters on the operation to generate a radiofrequency stimulation pulse, including but not limited to duty cycle, amplitude, and duration of radiofrequency stimulation pulses. In one example, the radiofrequency stimulation pulse patterns may be pre-defined and delivered to the electrode(s) to generate a desired stimulation depending on the user's requirement.

According to the invention, the low power consumption micro-controller contains a processor for controlling pulsed-radiofrequency stimulation. Preferably, the micro-controller is designed to have a considerably smaller size than conventional stimulation electronic stimulator so that they may be implanted into a subject. For example, the micro-controller may be configured in a chip, such as a bio-chip, which may be made from any implantably acceptable material. The amplitude as needed is very low, such as less than 20 volts, preferably less than 10 volts. In one example of the invention, the amplitude as need is in a range from +10 to −10 volts, preferably from +5 to −5 volts; the stimulus pulse train at an RF of 500 KHz frequency with pulse rate of 2 Hz, and the duration time of 300 seconds. The PRF waveform used in the invention may be a monophasic retangluar pulse shape, a bi-phasic pulse shape, a sinusodial or triangular pulse shape. In one example of the invention, a bi-phasic PRF waveform is used to maintain charge balance. In another example of the invention, a sinusodial or triangular PRF waveform is used for optimal effectiveness.

According to the invention, the micro-controller is implanted into the body of the subject, such as under the skin, and should be placed at an appropriate location near the nerve or tissue to be treated so that the electrode(s) can be exposed on or around the nerve or tissue to be treated. The micro-controller delivers the electronic stimulation pulse patterns to the electrode(s) via a wire. For instance, in the stimulator for back pain therapy according to the invention, the micro-controller may be placed around the lumbar region in the body of the subject.

According to the invention, the electrode(s) may be in the form of two electrodes, or one electrode which is configured as a uni-polar with a long return path, or a bipolar or multiple-polar electrode with a short return path, or a means for generating multiple stimulations with multiple contact electrodes, such as a lead or a stick having multiple contact electrodes. In one example of the invention, the electrode(s) can be extended into a multiple electrode array for large area or multipoint applications. In one example of the invention, two electrodes are used. In another example of the invention, a bipolar electrode is used. According to the invention, the stimulation pattern may be defined before the application. For instance, a lead with two or more contact electrodes is used, which delivers a pre-defined electrical stimulation patterns to the desired location.

According to the invention, the electrode(s) should be placed at an appropriate location on or around the nerve or tissue to be treated. For instance, the electrode(s) may be positioned an appropriate location in the body of the subject through any of imaging technologies, for example, fluoroscope, computed tomography (CT), magnetic resonance imaging (MRI) and ultrasound guided technologies, or a non-imaging navigation system such as global positioning system (GPS), magnetic field, endoscope guided visualization and the like, or a combination thereof.

According to the invention, the electrode(s) may be fixed to the nerve(s) or tissue(s) with a fixing device, such as anchors, bio-glue, bio-mimetic adhesives (“gecko tape”), bio-materials for immobilization or any other setting mechanism for fixing the electrode(s) to the appropriate location(s) as desired. For example, the electrode(s) may be fixed into the tissues, such as muscle(s), ligament(s), bone(s) or cartilage(s), surrounding the nerve(s) or tissue(s) to be treated. Referring to FIG. 2 which shows a particular example of the invention providing a stick with multiple electrodes containing one polar electrode on the top of the stick and eight contact electrodes positioned on the body of the stick, the stick has four anchors that are extended from the stick and controlled by an on/off switch. In this particular example, the four anchors will be extended from the stick after the electrode is implanted into the body and located at an appropriate location as desired, and the anchors are fixed to the cartilages and/or muscle surrounding the nerve or tissue to be treated.

According to the invention, the stimulation system is battery-less to make it possible to develop a relatively small-sized stimulator. In a preferable embodiment of the invention, a remote charger for power supply is used in the stimulation system. The remote charger may be a near field inductive coupling, or any other remote charging technologies for power supply with output regulator circuit, e.g. wireless charging technologies including but not limited to electromagnetic induction coupling or resonate inductive coupling, or capacitive coupling, or light (optical, laser) or Radio frequency (RF) spectrum (such as 900 MHz band or radio or microwave) charging system. For instance, a Class-E power amplifier may be used for power supply outside the body. In a more preferable example of the invention, the stimulator is implanted under the skin around the back, and the controller is recharged by a pair of coupled coils through the skin.

Furthermore, the stimulation system of the invention may comprise a means for measuring one or more functional or physiological indicators such as temperature of the nerve or tissue surrounding the means for generating a stimulation such as the electrode(s), which may be loaded on the electrode(s), and/or a transmitter for transmitting out the signals including the functional or physiological indicators or the working parameters of radiofrequency stimulation pulses, which may be configured in the micro-controller.

In one embodiment of the invention, the stimulation system comprises an external controller for receiving displaying and/or transmitting one or more functional or physiological indicators of the nerve or tissue surrounding the electrode(s), or the temperatures of the micro-controller, and one or more electrical stimulation parameters such as duty cycle, frequency, amplitude, duration, pulse frequency, and waveform. In one example of the invention, the external controller comprises a receiver for receiving the signals from the transmitter, a displayer or a recorder for displaying and/or recording the signals or parameters, and/or a means for transmitting the commends on the electrical stimulation parameter patterns to the micro-controller. Programmable parameters may be adjusted in accordance with the transmitted information received by the receiver, and used for controlling electronics to modify the generation of the pulse.

In one example of the invention, the stimulation system comprises an external controller, and a means for measuring one or more functional or physiological indicators such as temperatures of the surrounding tissue, which is configured in the electrode(s), and a transmitter for transmitting the signals to the external controller and a receiver for receiving the commends from the external controller, both of which are configured in the micro-controller.

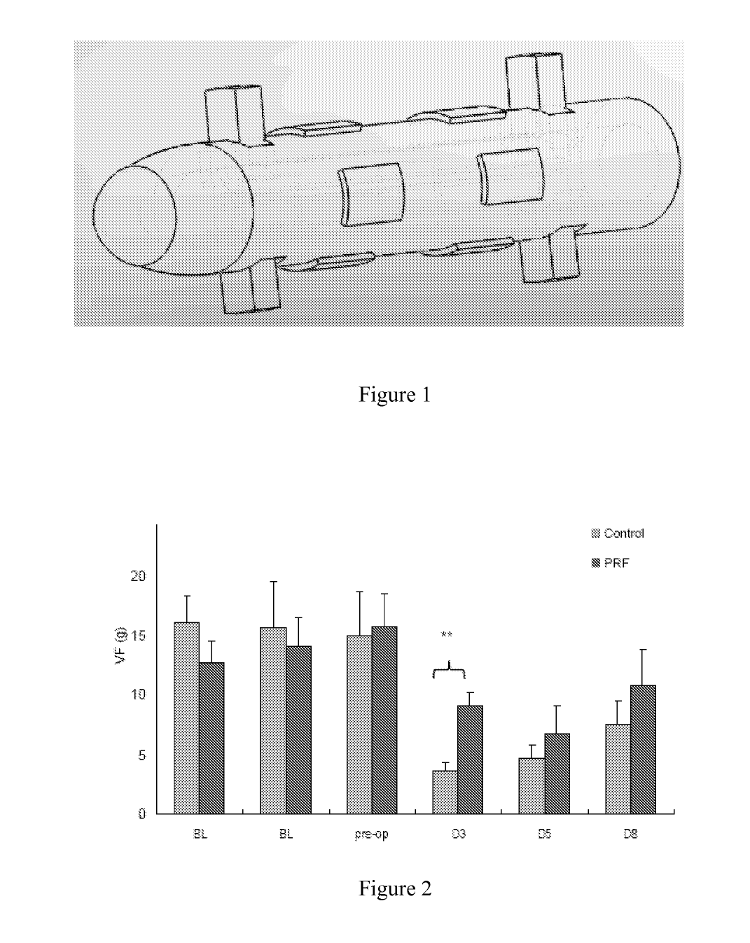

In one embodiment of the invention, a system block diagram of the proposed CMOS SoC comprises a micro-controller configured in a chip and a bi-polar electrode is implanted into the body of the subject. Referring to FIG. 1, the micro-controller contains a radio frequency to direct current (RF-DC) circuit, a voltage limiter, a low dropout regulator (LDO), an RF receiver, a clock regenerator, a logic controller and an PRF driver. The RF-DC circuit receives power from an external 1 MHz RF power source outside the skin. This circuit converts the RF signal into a DC voltage. The following voltage limiter limits the DC voltage to a maximum of 5V, which can be regulated by the LDO to 1.4-3.3V. The clock regenerator extracts the clock signal from the RF source for the logic controller, which generates default bi-phasic PRF waveforms for the PRF drivers. The bi-phasic outputs are delivered to a pair of bi-polar electrodes through two coupling capacitors for charge balance. Both the electrodes are exposed into the surgically exposed L5 nerve of the lumbar region for stimulus. Furthermore, the RF on-off keying (OOK) receiver receives external commands from an external controller such as a personal computer (PC) or a personal data assistant (PDA) and directs the logic controller to output the specified PRF waveform. The power is supplied by a Class-E power amplifier via coils, and the commands from the external controller are received by the receiver, and transmitted to the logic controller to drive a pulsed-radio frequency stimulation through the bipolar electrodes. This implantable SoC uses 402 MHz command signals following the medical implanted communication system (MICS) standard and a low frequency (1 MHz) coil size for easy user alignment and increased penetration depth. In addition to the default parameters (a pulse train with a period of 0.5 sec modulated by a 500-KHz carrier), the user can specify a custom stimulation protocol in the logic controller via a handheld device. The RF power is inductively coupled to a coil antenna and converted to DC by a full-wave rectifier consisting of 4 diode-connected metal oxide semiconductor (MOS) transistors. For heat reduction, the bodies of 2 PMOS transistors are floating, such that the rectified current does not go through the PN junctions in the substrate and, hence, the reverse recovery current, which causes additional power loss, is avoided. The bodies of the 2 NMOS devices are weakly tied to the ground by the substrate resistor for the same purpose. The clock regenerator, which is a Schmitt trigger circuit, regenerates the 1 MHz clock. The bi-phasic pulse train outputs are obtained by splitting the signal path into two branches, one with an inverter and the other without an inverter. The two PRF drivers, each consisting of three cascaded inverters that increase driving capability, can generate output voltages in the range of ±1.4V to ±3.3V through off-chip coupling capacitors.

The SoC chip is fabricated in a 0.35 nm CMOS process and mounted on a printed circuit board (PCB), which is connected to a flexible coil antenna. This DRG stimulator module is as small as a US quarter. The measured efficiency of the RF-DC circuit is 80%.

When connected to 10 k and 50 k load resistors, the PRF driver delivers an output power of 0.37 mW and 9.5 mW respectively. PRF waveforms with different periods (0.05 to 1.25 s) and different modulation frequencies (4 to 1000 kHz) can be measured successfully. Powered by the external power source, the SoC with a 10 k load dissipates 12.48 mW and has a chip temperature of <39° C., as measured by Infrared (IR) thermography. Assuming the same tissue impedance, the power level needed for nerve stimulation is much lower (roughly 1/12) than that using the conventional method. It is believed that the invention the only batteryless, SoC-based implantable stimulator among the known stimulation systems. The therapeutic efficacy of the invention was confirmed by an animal study.

Animal Study

An animal study on neuropathic pain model was conducted following the Von Frey experiment as described in a text book, such as “Mechanisms of Neuropathic Pain”, edited by James N. Campbell1,* and Richard A. Meyer1, 2006.

Animal Preparation

Male Sprague-Dawley rats (250-300 g, National Laboratory Animal Center, Taiwan) were used in this animal study. The rats were housed in groups of two to five per cage and acclimatized to the laboratory conditions (12-h light/dark cycle; 22±1° C. room temperature). All animals had free access to food and water.

Surgical Procedure and PRF Treatment on DRG

All the rats were performed with isoflurane (4% to induce; 1.5-2% to maintain) in air delivered via a nose cone. To perform a spinal nerve ligation (SNL), the L5 spinal nerve was isolated and tightly ligated with 6-0 nylon thread. A complete hemostasis was confirmed. The electrode was connected to an PXI-5401 Function Generator (National Instruments, USA) to generate the pulsed radiofrequency lesion, The rats were randomly assigned to two groups after the surgical procedure: the control group (n=5) and the treatment group (n=6). After the surgical procedure of SNL, the rats of the treatment group were treated with a PRF stimulation by a bi-polar electrode correctly located on the dorsal root ganglion of the L4-L5 foramen with the parameters of ±5 volts, 500-KHz RF pulses, 25 milliseconds in duration according to the invention. The pulses were delivered at a rate of 2 Hz for a period of 5 minutes.

Behavioral Experiment

All the rats were habituated to the testing environment from Day 1 before the baseline testing. For mechanical stimulation, the rats were individually placed in plastic boxes (10×10×10 mm) on an elevated mesh floor and allowed to acclimate for 15 minutes before the threshold testing. Mechanical thresholds after SNL surgery were determined using von Frey filaments. PRF Stimulations were conducted in a consecutive fashion, ascending or descending. The 50% withdrawal threshold was determined as a VF value. The animals were tested daily from Day 1 before surgery, and Days 3, 5, and 8 after the surgery.

The mechanical thresholds in terms of VF values of the base line (BL) and that measured before the surgery (pre-op), and after the surgery were shown in FIG. 2. The results shown in FIG. 2 demonstrated that the rats of the control group after SNL had a significant reduction of mechanical withdrawal threshold to von Frey stimulation; but the rats of the treatment group did not. The averages of the VF values of the control group were less than those of the treatment group at Days 3, 5 and 8 after surgery, In particular, it was found that there was a significant improvement (P<0.001) between the treatment group (average: 9.10±1.15) and the control group (average: 3.72±0.58) at Day 3 after the surgery.

In view that the treatment group consistently had higher pain tolerance than the control group, it was concluded that the treatment with a PRF stimulation at a low amplitude on the DRG according to the invention was effective in treatment of pain.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

I/we claim:

1. An implantable pulsed-radiofrequency stimulator for treating a nervous symptom or condition, which comprises:
a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the controller via a wire for delivering the electrical stimulation pattern.

2. The stimulator of claim 1, wherein the nervous symptom or condition is selected from the group consisting of chronic pain, motor disorder, cognitive disorder, obesity, epilepsy, depression, incontinence, sexual dysfunction, back pain, tremor, dystonia, spasticity, Parkinson's disease, urinary and fecal incontinence.

3. The stimulator of claim 1, wherein the electrode(s) is(are) exposed on or around an appropriated location of the central or peripheral nervous system as desired, including vegus nerves, gastric nerves, motor nerves, cortex tissues, or deep brain.

4. The stimulator of claim 3, wherein the electrode(s) is(are) exposed on or around a dorsal root ganglion or a spinal ganglion of spine or a trigeminal ganglion of the 5^{th} cranial nerves, or basal ganglia, hippocampus of brain, cerebellum, or autonomic nerve, or peripheral nerves.

5. The stimulator of claim 2, wherein the nervous symptom is back pain.

6. The stimulator of claim 5, wherein the electrode(s) is(are) exposed on or around a dorsal root ganglion.

7. The stimulator of claim 1, wherein the working parameters of radiofrequency stimulation pulses comprise duty cycle, amplitude, and duration of radiofrequency stimulation pulses.

8. The stimulator of claim 1, wherein the low power consumption micro-controller contains a processor for controlling pulsed-radiofrequency stimulation.

9. The stimulator of claim 1, wherein the low power consumption micro-controller is configured in a chip.

10. The stimulator of claim 1, wherein the amplitude as needed is less than 20 volts.

11. The stimulator of claim 10, wherein the amplitude as needed is the amplitude as need is in a range from +10 to −10 volts.

12. The stimulator of claim 1, wherein the PRF waveform is a monophasic retangluar pulse shape, a bi-phasic pulse shape, a sinusodial or triangular pulse shape.

13. The stimulator of claim 1, wherein the electrode(s) is(are) in the form of two electrodes, or one electrode which is configured as a uni-polar with a long return path, or a bipolar or multiple-polar electrode with a short return path, or a stimulation mode with multiple contact electrodes.

14. The stimulator of claim 13, wherein the electrode(s) is(are) in the form of a bipolar electrode.

15. The stimulator of claim 1, wherein the electrode(s) is(are) positioned at the desired location through imaging technologies or non-imaging navigation system, or a combination thereof.

16. The stimulator of claim 1, wherein the electrode(s) is(are) positioned at the desired location through fluoroscope, computed tomography (CT), magnetic resonance imaging (MRI), global positioning system (GPS), magnetic field, endoscope guided visualization.

17. A stimulation system for treating a nervous symptom or condition, which comprises:
a remote charger for power supply; and
an implantable pulsed-radiofrequency micro-stimulator comprising a low power consumption micro-controller for controlling the working parameters of radiofrequency stimulation pulses by providing a radiofrequency stimulation pattern; and
at least one electrode for generating a pulsed-radiofrequency stimulation at a low amplitude, which is connected with the controller via a wire for delivering the electrical stimulation pattern according to claim 1.

18. The system of claim 17, wherein the remote charger for power supply is a near field inductive coupling, an electromagnetic induction coupling a resonate inductive coupling, or a capacitive coupling, or a light or Radio frequency (RF) spectrum charging system.

19. The system of claim 17, wherein the remote charger for power supply is a Class-E power amplifier.

20. The system of claim 17, further comprising an external controller for receiving, recording, processing, displaying or transmitting one or more functional or physiological indicators, or electrical stimulation parameters.

21. The system of claim 17, further comprising a means for measuring one or more functional or physiological indicators of the tissue or nerve surrounding the electrode(s), which is(are) loaded in the electrode(s).

22. The system of claim 17, wherein the micro-controller comprises a transmitter for transmitting the signals to the external controller, a receiver for receiving commends from the external controller.

23. The stimulator of claim 1, wherein the electrode(s) is(are) fixed into the tissues surrounding the nerve(s) or tissue(s) with anchors.
